(19) United States
(12) Patent Application Publication
(10) Pub. No.: US 2012/0316462 A1
(43) Pub. Date: Dec. 13, 2012

(54) STOOL SPECIMEN COLLECTION SYSTEM

(75) Inventors: Joellyn Enos, Arena, WI (US); Hemanth Shenoi, Verona, WI (US); Scott Castanon, Carlsbad, CA (US); Keith Kopitzke, Fallbrook, CA (US)

(73) Assignee: EXACT SCIENCES CORPORATION, Madison, WI (US)

(21) Appl. No.: 13/491,983

(22) Filed: Jun. 8, 2012

Related U.S. Application Data
(60) Provisional application No. 61/495,847, filed on Jun. 10, 2011.

(51) Int. Cl. A61B 10/02

(52) U.S. Cl. 600/562

(57) ABSTRACT

Provided herein is technology relating to collecting samples and particularly, but not exclusively, to technology for collecting a stool specimen.

The present application claims the benefit of U.S. Provisional Patent Application Ser. No. 61/495,847, filed Jun. 10, 2011, which is incorporated by reference in its entirety.

### FIELD OF INVENTION

Provided herein is technology relating to collecting samples and particularly, but not exclusively, to technology for collecting a stool specimen.

### BACKGROUND

In the medical clinic, laboratory examination of fecal samples is an important component of some diagnoses. In these cases, stool specimens are taken from a patient and examined for conditions relative to the ailment of the patient. As specific examples, physicians and clinicians often test stool samples as a component of testing for colon or rectal cancer or to identify bacteria or viruses that may be involved in an infection. Additionally, some diagnostics require isolating and assaying nucleic acids, proteins, fats, or other analytes present in a stool sample. Consequently, acquiring a specimen for testing (e.g., a stool specimen) is the first step in sample processing for many tests (e.g., the medical diagnostic analysis of feces). Acquiring the specimen presents several challenges relating to, e.g., protecting those who produce and handle the specimen from exposure to the specimen, minimizing contamination of the specimen, and providing for the sanitary disposal and/or easy cleaning of materials that contact the collected specimen.

Solutions for addressing problems associated with collecting a stool specimen include devices comprising a paper catchment area attached to a frame and/or other support. The devices are designed to be used on a toilet or commode so that the frame places the catchment over and within the toilet bowl and allows the subject to position itself on the toilet seat in a manner similar to that of its usual operation. The stool specimen is deposited into the catchment area and an analytical stool sample is taken for testing. After removing the portion for sampling, the paper bowl and any remaining contents may be released into the toilet for sanitary disposal. Conventional solutions comprise designs that are awkward to use. For example, release of the portion of the device receiving the sample, e.g., a bowl, often requires fine motor movements and decent vision, conditions that do not apply to a large proportion of the subjects who require medical testing and use the conventional solutions to collect a specimen. As a consequence, extant solutions present a significant risk of the user contaminating the sample or touching the unused stool portion when attempting disposal. Also, some conventional solutions mount on rather than below a toilet seat, thus increasing the user's discomfort and/or the likelihood that the user will accidentally contact or spill the specimen during use. Accordingly, improved devices are required that allow for the comfortable acquisition of a stool specimen, that are easy to use for the subjects providing stool specimens, that include protections for minimizing contact between a stool specimen and the subject, and that facilitate easy, sanitary disposal.

### SUMMARY

Provided herein is technology relating to collecting samples and particularly, but not exclusively, to technology for collecting a stool specimen. In embodiments of the device, the device comprises a bowl secured to a frame by a removable retainer. After deposition of stool, the retainer is removed to release the bowl for disposal. Embodiments of the device are provided in various configurations that provide for the collection of a stool specimen and embodiments are provided in which the components are made from various materials. For example, in some embodiments the frame is made from a rigid plastic, the bowl is made from paper, and the retainer is made from rubber.

Accordingly, provided herein is a device for collecting a stool specimen. The device comprises a frame comprising a top side, a bottom side, and a hole; a bowl adapted to fit the hole; and a removable retainer to secure the bowl to the frame at the hole, wherein the bowl protrudes from the frame on the bottom side and the removable retainer is operable from the top side. With respect to the attachment of the bowl to the frame, in some embodiments the hole has a perimeter and the bowl is secured to the frame along essentially the entire perimeter of the hole. In some embodiments, the bowl is disposable, flushable, and/or biodegradable. For example, in some embodiments the bowl is made of paper. The device is designed to collect a stool specimen from a subject; thus, in some embodiments the frame is adapted to fit on a toilet and in some embodiments the frame is adapted to be placed on a toilet bowl under a toilet seat. Some embodiments provide that the frame is foldable. The frame is not limited in the materials that are used in its construction. For example, in some embodiments the frame is made of plastic. In some embodiments the frame comprises a marking, e.g., a marking designating the front of the device.

The device provides a retainer for securing the bowl to the frame and that is removable for release of the bowl for disposal. The device is designed for easy use by a user such as a subject. As such, in some embodiments the retainer is adapted to be removed by a single action, e.g., one pull. It is to be understood that the technology is not limited in the manner in which the bowl is secured to the frame by the retainer. For instance, in some embodiments the retainer secures the bowl to the frame by friction and in some embodiments the retainer exerts a force upon the bowl to secure it to the frame. Moreover, the retainer is not limited in its design, shape, or in the materials from which it is made, provided it functions for the purposes essentially as described herein. For example, in some embodiments the retainer comprises a band, a cord, or a U-channel. In some embodiments the retainer comprises a pull tab adapted for a user to pull with a hand to remove the retainer. The pull tab is not limited in its shape, e.g., embodiments provide a pull tab comprising a T-shaped handle, a round tab, or a ring. In some embodiments the pull tab comprises a marking such as “PULL”. Also, in some embodiments the retainer further comprises an adhesive. In some embodiments the retainer is made of solid rubber, foam rubber, cardboard, or plastic. Like the bowl, some embodiments provide a retainer that is disposable. For example, in some embodiments the retainer is flushable and/or biodegradable.

The designs of the frame and bowl are not limited in their shape provided the bowl is secured to the frame and can be released in accordance with the technology described. For example, in some embodiments the hole is round and in some embodiments the hole is square, oval, rectangle, diamond, pear-shaped, or teardrop-shaped.

The technology also provides methods for collecting a stool sample. Thus, it is appropriate that some embodiments of the methods comprise placing a device as described above on a toilet and depositing a stool sample into the bowl. In some embodiments the method comprises placing the device on a toilet bowl and under a toilet seat. Embodiments of the methods comprise further steps including, for example, taking an analytical sample from the deposited stool specimen and/or disposing of the bowl. In some embodiments disposing of the bowl comprises removing the retainer.

The technology provided herein finds use in related systems for collecting a stool specimen. These systems comprise a bowl functionality to collect a stool specimen, a frame functionality to support the bowl, and a removable retainer functionality to secure the bowl to the frame. In some embodiments removing the retainer functionality releases the bowl from the frame. It is to be understood that embodiments of the systems provided comprise any embodiments of the devices described herein.

In addition, the technology finds use in embodiments of kits, for example kits comprising any of the devices described herein. In some embodiments, the kits further comprise an instruction for use; storage, shipping, or protective packaging; and/or a solution. For example, in some embodiments the solution comprises a buffer, salt, preservative, and/or stabilization agent. The solution may be in a lyophilized form. The collected stool, in some embodiments, is further processed, e.g., in an analytical or diagnostic test. Accordingly, additional embodiments provide kits further comprising a sampling device and kits further comprising an analysis reagent or a detection reagent.

Additional embodiments will be apparent to persons skilled in the relevant arts based on the teachings contained herein.

### BRIEF DESCRIPTION OF THE DRAWINGS

These and other features, aspects, and advantages of the present technology will become better understood with regard to the following drawings:

FIG. 1A is a drawing of an embodiment of a stool specimen collection system in which the bowl is secured to the frame by a band retainer. FIG. 1B is a detail view of the retainer pull tab and shows the band retainer securing the bowl to the frame.

FIG. 2A is a drawing of an embodiment of a stool specimen collection system in which the bowl is secured to the frame by a cord retainer. FIG. 2B is a detail view of the retainer pull tab and shows the cord retainer securing the bowl to the frame.

FIG. 3A is a drawing of an embodiment of a stool specimen collection system in which the bowl is secured to the frame by a U-channel retainer. FIG. 3B is a detail view of the retainer pull tab and shows the U-channel retainer securing the bowl to the frame.

FIG. 4A is a top perspective view drawing of an embodiment of a stool specimen collection system in which the frame is foldable. FIG. 4B is a bottom perspective view drawing of the same embodiment.

FIG. 5 is a series of drawings of retainer pull tabs. FIG. 5A is a drawing of a T-shaped pull tab. FIG. 5B is a drawing of a round pull tab. FIG. 5C is a drawing of a ring pull tab.

### DETAILED DESCRIPTION

Provided herein is technology relating to collecting samples and particularly, but not exclusively, to technology for collecting a stool specimen.

Definitions

To facilitate an understanding of the present technology, a number of terms and phrases are defined below. Additional definitions are set forth throughout the detailed description.

As used herein, “a” or “an” or “the” can mean one or more than one. For example, “a” widget can mean one widget or a plurality of widgets.

As used herein, the term “analyte” is to be construed broadly as any compound, molecule, element, ion, or other substance of interest to be detected, identified, or characterized.

As used herein, the term “bowl” refers to a receptacle having, when secured to a frame at the hole therein, an interior space or concavity configured to receive a stool from a subject during use. The term “bowl” is not intended to limit the receptacle to any particular shape or configuration and may be, for example, generally hemispherical, cylindrical, conical, rectilinear, ellipsoid, etc.

As used herein, the term “flushable” means suitable for flushing down a toilet.

As used herein, the terms “subject” and “patient” refer to an animal, preferably a human, from which a stool specimen is collected. In some instances, the subject is also a “user” (and thus the user is also the subject or patient).

As used herein, the term “removable retainer” refers to a retainer that is movable from a position in which it effects securing the bowl to the frame to a position in which the bowl is released from the frame. In some embodiments, the removable retainer is reversibly affixed to the frame, e.g., by friction, while in position to secure the bowl to the frame, but is not otherwise attached to the frame. In other embodiments, the removable retainer may comprise an irreversible attachment to the frame such that it remains attached to the frame after release of the bowl.

### Embodiments of the Technology

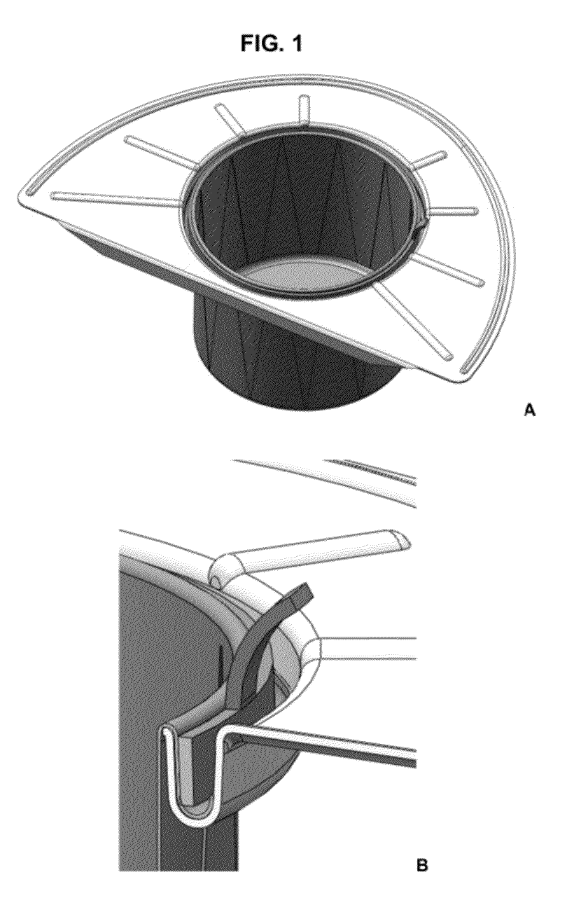

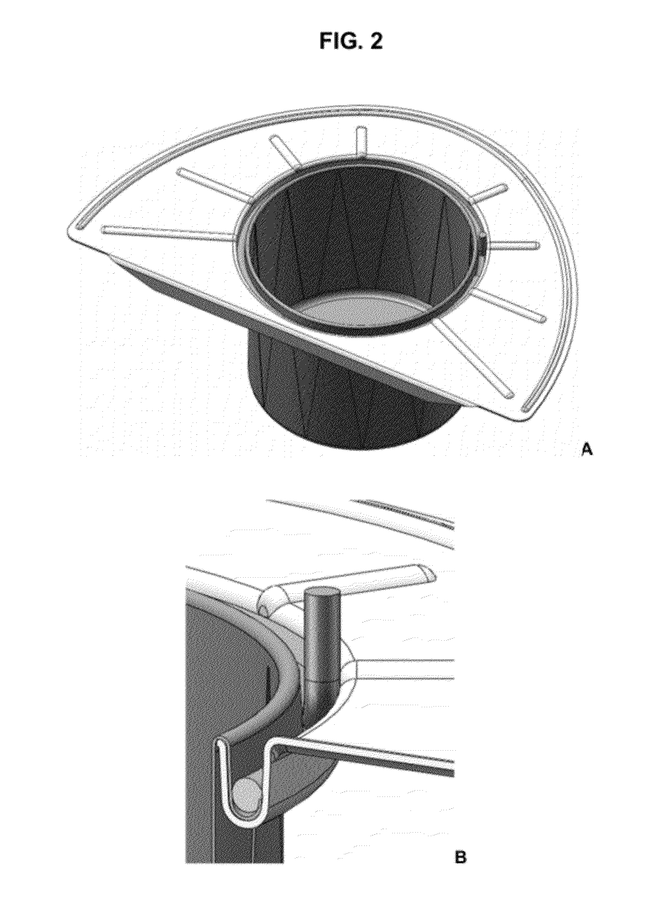

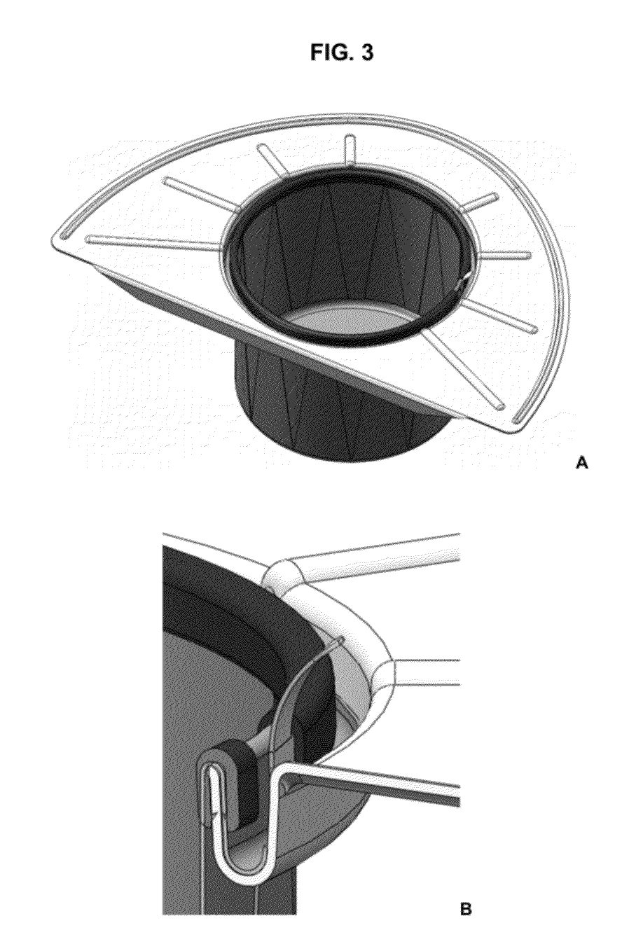

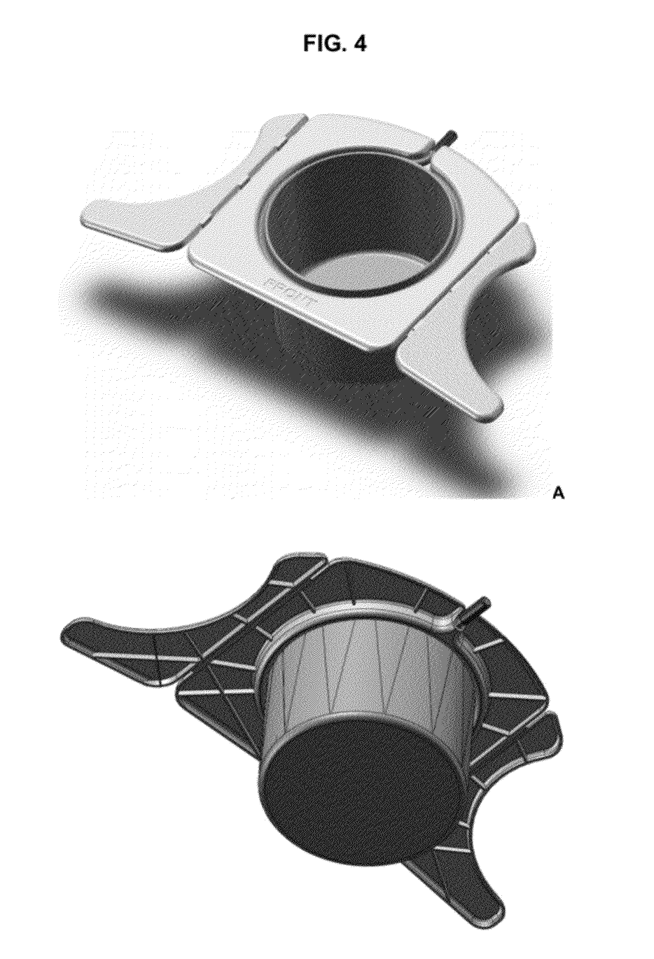

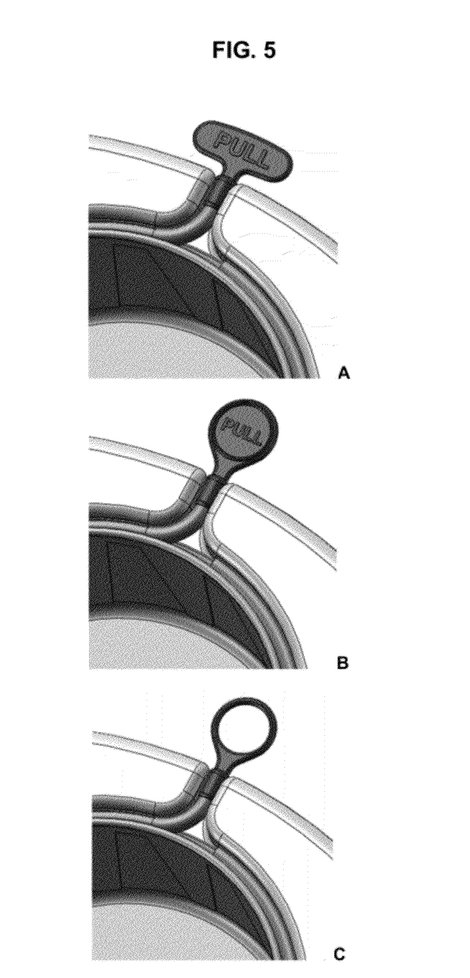

The technology provided herein relates to collecting stool specimens. The technology includes and contemplates devices and related embodiments such as those embodiments portrayed in FIGS. 1-5. In these embodiments, a specimen collector comprises a bowl secured to a frame by a retainer. The device is designed in some embodiments to be placed on a toilet bowl and under the toilet seat. This design helps hold the device in place for deposition of the stool specimen and allows the subject to sit on the toilet seat in the same manner as during normal toilet use (e.g., as a subject would use a toilet without the collection device). After depositing the specimen into the bowl, an analytical sample may be taken for testing and, optionally, the bowl and remaining contents may be released from the frame for disposal (e.g., by removing the retainer).

In some embodiments, the bowl is disposable (e.g., the bowl is flushable, for example, in a toilet, and/or the bowl is biodegradable) and in some embodiments the bowl is made of paper. In some embodiments the bowl comprises a coating, for example, a water-resistant coating to prevent or minimize absorption of fluids by the bowl when the bowl is contacted by water and/or other fluids or liquids that could damage the bowl and/or compromise its structural integrity. The technology, however, is not limited in the materials that are used for the bowl. For example, other materials are appropriate as long as they meet the needs of the technology, e.g., the bowl is made in some embodiments from plastic, cardboard, glass, fabric (e.g., woven and/or comprising a natural or synthetic fiber), foam, etc. One of skill in the art can identify materials that are appropriate for flushable, disposable, and biodegradable embodiments.

The frame provides a rigid support for the bowl to facilitate collection of the stool specimen in the bowl. The frame comprises a hole, the perimeter of which mates with the perimeter of the opening of the bowl. The bowl is attached on the bottom side of the frame such that, when the device is placed on a toilet for use with the top side facing up, stool from a subject passes through the hole into the attached bowl. Though embodiments of the frame are made from plastic, other materials are appropriate for its construction such as, e.g., metal, wire, wood, cardboard, foam, and the like. In some embodiments the frame is foldable and in some embodiments the frame and/or bowl comprise markings to direct the assembly and/or use of the collection device. For example, embodiments of the frame comprise markings such as “FRONT” to orient the device relative to the user and/or the toilet on which the device is placed. The frame is suitable in some embodiments for repeated use by attaching a new bowl to it for each individual use. It is contemplated that the frame, accordingly, is made from washable (e.g., soap- and/or chemical-resistant) and/or sterilizable (e.g., chemical-resistant, autoclavable, UV-resistant, ozone-resistant, etc.) materials to prevent or minimize degradation of the frame through repeated cycles of use, cleaning, and sterilization.

The retainer secures the bowl to the frame and is removable from the top side of the device (e.g., the side opposite the attached bowl). In use, e.g., after a sample is taken from the stool specimen deposited into the bowl, a user removes the retainer from the device to release the bowl (and any remaining contents, if present) from the frame for disposal (e.g., the bowl is released into the toilet bowl for flushing). In some embodiments, it is contemplated that a single pull of the retainer releases the bowl. In some embodiments, the retainer is wedged between two surfaces to secure the bowl to the frame by friction and/or by exerting a force upon the bowl to press it against the frame. The retainer holds the bowl in place sufficiently to hold a heavy load of stool while also being sufficiently easy to remove by the user of the specimen collector (e.g., the subject in some embodiments). It is contemplated that the retainer takes any shape that can provide for a secure assembly of the bowl and frame while also being easy to remove. For example, in some embodiments the retainer is a band, a cord, a U-channel, etc. In some embodiments the retainer comprises a pull tab or handle to facilitate its removal from the device and in some embodiments the pull tab or handle comprises a marking to direct the user with respect to use of the retainer (e.g., a marking such as “PULL” on the pull tab). In some embodiments, the pull tab increases the distance between the specimen and the user when the retainer is operated, thus further minimizing the possibilities of exposing the sample to contamination by the user and exposing the user to the specimen. Several embodiments of the pull tab are contemplated for manual manipulation including, for example, a T-shaped handle, a round tab, a ring, etc. The retainer is made from a material suitable for use of the device as described. For example, the retainer in some embodiments is made from rubber, foam, plastic, other elastomers, wire, woven fiber, cardboard, and the like. In some embodiments the retainer comprises an adhesive. In some embodiments the retainer is made from disposable materials so that the user can dispose of the retainer with the bowl (e.g., by flushing the bowl and retainer in a toilet). Accordingly, it is contemplated that the retainer in such embodiments is made, e.g., from a flushable and/or biodegradable material.

The device is not limited in the shape of the components provided they fit together and operate as described. In some embodiments the bowl comprises a round opening and the frame comprises an appropriately shaped (e.g., round) hole onto which the bowl fits. Other contemplated embodiments comprise bowl openings that are other shapes, e.g., oval, square, rectangle, diamond, pear-shaped, or teardrop-shaped, etc., and frames comprising coordinately shaped holes. The retainer is of a size, shape, and material appropriate to secure the bowl in the embodiments contemplated.

The devices provided are associated with related methods. For example, a method for obtaining a stool specimen includes the steps of placing the device on a toilet bowl, lowering the toilet seat onto the device to hold it in place, and then depositing a stool specimen into the bowl of the device. Additional optional steps include taking an analytical sample, in some embodiments through use of a sampling device (e.g., as provided in U.S. Patent Application Ser. Nos. 61/476,707, and 13/449,878, each of which is incorporated herein by reference in its entirety for all purposes), and disposing of the bowl (e.g., without touching the bowl or its contents) and/or any remaining stool and/or the retainer.

Also provided herein are systems for collecting a stool specimen comprising a bowl functionality to collect a stool specimen, a frame functionality to support the bowl, and a retainer functionality to secure the bowl to the frame. Embodiments of the systems comprise the variations and modifications described for the devices and methods provided above as well as those embodiments one of skill in the art recognizes to be encompassed by the scope of the description provided herein.

The technology finds use in kits comprising embodiments of the devices and systems described and, in some embodiments, optional components such as, e.g., an instruction for use (e.g., providing the steps of a related method) and any related packaging for storage, shipping, and the like. Embodiments of the kits may comprise one or more solutions, e.g., comprising a stabilization reagent, a buffer, a salt, or a preservative for using with (e.g., for treating, homogenizing, preserving, or storing) the collected specimen and the analytes it contains. Kits may also comprise other components useful for sample processing such as sampling devices and homogenizers and/or components for testing the sample such as analysis reagents and/or detection reagents, etc.

Although the disclosure herein refers to certain illustrated embodiments, it is to be understood that these embodiments are presented by way of example and not by way of limitation. All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in engineering, material science, pharmacology, biochemistry, medical science, or related fields are intended to be within the scope of the following claims.

We claim:

1. A device for collecting a stool specimen, the device comprising:
a) a frame comprising a top side, a bottom side, and a hole;
b) a bowl adapted to fit said hole; and
c) a removable retainer to secure said bowl to said frame at said hole,
wherein said bowl protrudes from the frame on said bottom side and wherein said removable retainer is operable from said top side.

2. The device of claim 1 wherein said hole has a perimeter and wherein said bowl is secured to said frame along essentially the entire perimeter of said hole.

3. The device of claim 1 wherein the bowl is flushable.

4. The device of claim 1 wherein the bowl is made of paper.

5. The device of claim 1 wherein the frame is adapted to fit on a toilet.

6. The device of claim 1 wherein the frame is foldable.

7. The device of claim 1 wherein the retainer secures the bowl to the frame by friction.

8. The device of claim 1 wherein the retainer comprises a band, a cord, or a U-channel.

9. The device of claim 1 wherein the retainer is made of rubber, cardboard, woven fiber, or plastic.

10. The device of claim 1 wherein the retainer is flushable.

11. A system for collecting a stool specimen comprising:
a) a bowl functionality to collect a stool specimen;
b) a frame functionality to support the bowl; and
c) a removable retainer functionality to secure the bowl to the frame.

12. The system of claim 11 wherein removing the retainer functionality releases the bowl from the frame.

13. A kit comprising the device of claim 1.

14. The kit of claim 13, further comprising an instruction for use.

15. The kit of claim 13, further comprising storage, shipping, or protective packaging.

16. The kit of claim 13 further comprising a buffer solution.

17. The kit of claim 16 wherein said buffer solution comprises a salt, a preservative, or a stabilization agent.

18. The kit of claim 13 further comprising a sampling device.

19. A method for collecting a stool sample, the method comprising:
a) placing a device on a toilet or commode, wherein said device comprises:
i) a frame comprising a top side, a bottom side, and a hole;
ii) a bowl adapted to fit said hole; and
iii) a removable retainer to secure said bowl to said frame at said hole,
wherein said bowl protrudes from said frame on said bottom side into said toilet or commode, and wherein said removable retainer is operable from said top side;
b) depositing a stool specimen into said bowl;
c) taking an analytical sample from said stool specimen;
d) removing said removable retainer from said device.

20. The method of claim 19, further comprising step e) disposing of said bowl into said toilet or commode.
